(19) United States
(12) Patent Application Publication
(10) Pub. No.: US 2024/0374404 A1
(43) Pub. Date: Nov. 14, 2024

(54) MARKING COILS FOR STENTS & OTHER THROMBECTOMY -TYPE DEVICES AND RELATED PROCESSES

(71) Applicant: RC Medical, LLC, Lake Forest, CA (US)

(72) Inventor: Karissa Jackson, Lake Forest, CA (US)

(73) Assignee: RC Medical, LLC, Lake Forest, CA (US)

(21) Appl. No.: 18/659,501

(22) Filed: May 9, 2024

Related U.S. Application Data
(60) Provisional application No. 63/500,943, filed on May 9, 2023.

(51) Int. Cl. A61F 2/88

(52) U.S. Cl. CPC A61F 2/885; A61F 2250/0097

(57) ABSTRACT

Improved systems using marking coils are attached to stents and flow diverters and related thrombectomy systems at ends by polymer/coil or directly and can be two/three/four or any number needed.

### CROSS-REFERENCE TO RELATED APPLICATIONS

The present application claims priority to U.S. Provisional Application No. 63/500,943, filed May 9, 2023, the contents of which are incorporated herein by reference in their entirety.

### FIELD OF THE INVENTION

The field of the invention is compositions, devices, and methods of use and manufacturing of stents and other thrombectomy-type devices, both conceptually and in terms of structure specifically focused upon their function) especially as it relates to surgical removal of a thrombus within a subject blood vessel. Those skilled in the art understand this definitional approach to embrace any related art used during procedures having for example antegrade blood flow, wherein consequences of previously engaged emboli becoming partially or fully disengaged are dire.

### OBJECTS AND SUMMARY OF THE INVENTION

The present inventions comprise novel enhanced marking systems for braided stents and/or self expanding devices having at least about two or more diameters. Modulating and baffling blood flow is marked better for different diameter stenting and capture means as shown in the figure in schematic & cartoon form along with known and innovative coverings and coatings for the subject devices, and materials and properties impacting flow dynamics linked to the solutions claimed herein and embraced within the scope of the instant application for United Staters Letters Patent as herewith first disclosed in any form or reduced to practice by this written description.

### BRIEF DESCRIPTION OF THE DRAWINGS

Various preferred embodiments are described herein with references to the drawings in which merely illustrative views are offered for consideration, whereby:

FIG. 1 shows a schematic of marking coils on different long loops of a braided stent, and how they are attached;

FIG. 2 likewise shows marking coils on different long loops; and

FIG. 3 likewise shows multiple marking coils.

Corresponding reference characters are not needed indicate corresponding components throughout the several views of the drawings. Skilled artisans will appreciate that elements in the figures are illustrated for simplicity and clarity and have not necessarily been drawn to scale. For example, the dimensions of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments of the present invention.

### DETAILED DESCRIPTION OF THE INVENTION

The present inventor has developed numerous way to use improved marking coils to enhance visibility and placement of medical devices. Expressly referenced and incorporated herein as if fully set forth, along with file histories and references are U.S. Pat. No. 10,398,777 (showing Seldinger techniques and updated catheter systems useful with the instant system); U.S. Pat. Nos. 9,808,359; 8,585,713; 8,545,514; 8,197,543; and 8,088,140—each of which depicts the plethoric stent-like devices and other coil-laden implants, particularly for endovascular usage. Likewise, the instant description includes information that may be useful in understanding the present invention. It is not an admission that any of the information provided herein is prior art or relevant to the presently claimed invention, or that any publication specifically or implicitly referenced is prior art.

As claimed below, solutions to the problem that during visibility is key during thrombectomy with antegrade blood flow, among other things, requires optimal visibility of subject from the mechanical devices. Blood flow has a water-hammer effect on devices and emboli which can break pieces of the markers, like emboli are sheared off or entirely dislodged the emboli from the capturing devices. Avoiding this until now dangerous progression is among the objects of the instant teachings, it is respectfully submitted, by enhancing placement and fixation of marking coils.

In most cases, the dislodged marking devices preclude full visualization and often require use of more devices. Likewise being dislodged causers strokes and vessel occlusions.

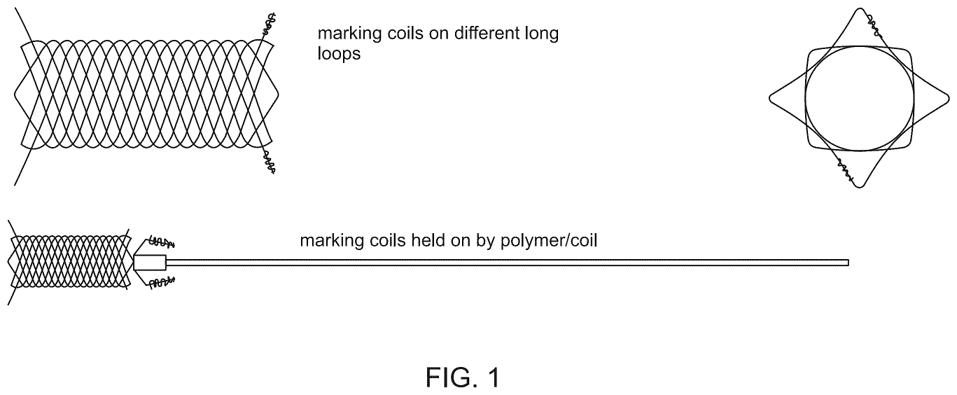

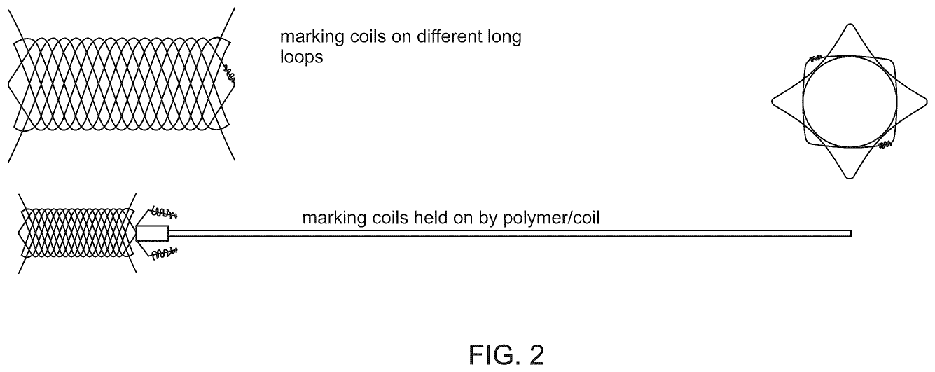

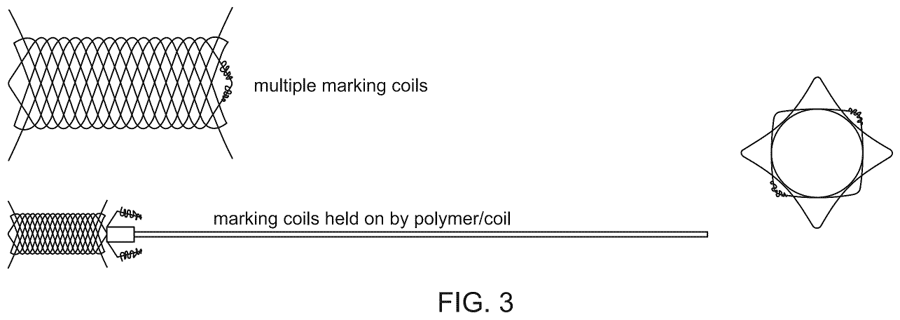

Referring now to FIG. 1, FIG. 2 and FIG. 3 marking coils are variously disposed about select long loops and secured by polymer coils, or not. Artisans understand that the various views, including straight on, and from the top indicate location of the improved marking systems for coils, in particular, an ideal example is the braided or woven stent. This type of stent has coils to which the marker of the present invention can be attached, and each of the Figures shows another and different way and located to attach, with and without supplemental polymeric attachments,

By way of further exemplary embodiments, the present inventive subject matter is drawn to compositions, devices, and devices/stents of thrombectomy-like including devices that have a mechanical self expanding device with 2 or more than 2 diameters, and other devices. Still referring both to FIG. 1-3, marking coils may be used with, for example Braided stents, such as with co-pending U.S. Ser. No. 16/988,698 (expressly incorporated herein as if fully set forth, including references) and the markers can be as shown in FIG. 1, on the long ends of the crown elements; or. As shown in FIG. 2 they may be located orthogonal to a plane bisecting the stent and may be held on with supplemental polymers. FIG. 3 shows placement on another device or flow-diverter, as claimed.

In addition the system can work with any other features, such as baffles, which can be covered with a material such as PTFE or polyurethan film or a braid of polymer of metal to further enhance the baffle properties of different diameters.

The illustrative “braided stent’ shown in the figures demonstrates that the markers can be more proximal and adjacent to the guidewire junction.

Also the distal tip can be configured with closed end or porous end. The distal tip can be closed with a laser cut configuration and/or covered with PTFE, or a braid of polymer or metal.

Various objects, features, aspects and advantages of the inventive subject matter will become more apparent from the following detailed description of preferred embodiments, along with the accompanying drawing figure.

The inventors have discovered that thrombectomy devices with improved functional characteristics, and especially with improved thrombus retention can be prepared by adjusting the diameters of the device.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used.

Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

Reference throughout this specification to “one embodiment” or “an embodiment” means that a particular feature, structure, or characteristic described in connection with the embodiment is included in at least one embodiment. Thus, appearances of the phrases “in one embodiment”, or “in an embodiment” in various places throughout this specification are not necessarily all referring to the same embodiment. Furthermore, the particular features, structures, or characteristics may be combined in any suitable manner in one or more embodiments.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar throughout this specification may, but do not necessarily, all refer to the same embodiment.

Furthermore, the described features, structures, or characteristics of the invention may be combined in any suitable manner in one or more embodiments. In the following description, numerous specific details are provided to provide a thorough understanding of embodiments of the invention. One skilled in the relevant art will recognize, however, that the invention may be practiced without one or more of the specific details, or with other methods, components, materials, and so forth. In other instances, well-known structures, materials, and so forth. In other instances, well-known structures, materials, or operations are not shown or described in detail to avoid obscuring aspects of the invention.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. An improved marking coil medical device system, which comprises, at least a structure including in combination:
at least a marking coil device affixed to a stenting member capable of being located on different long loops of the stenting member;
affixation accomplished via a secondary attachment mechanism or directly to the stenting member.

2. The improved marking coil medical device system of claim 1, wherein an at least two marking coils are attached on different or separate long loops.

3. The improved marking coil medical device system of claim 2, further comprising multiple marking coils.

4. The improved marking coil medical device system of claim 3, wherein the secondary attachment mechanism comprises a polymer coil.

5. The improved marking coil medical device system of claim 4, wherein there is no secondary attachment mechanism.

6. An improved marking system for stents, which comprises, in combination:
a first means for emplacement of marking coils, anywhere along the stent where traction is needed to a pusher element; and
a supplemental means for preventing movement between the stent in the catheter proximate to the dead space about the pusher.

7. The improved marking system of claim 6, being alternated and comprised of any and all locations about the stent, wherein maximum collapsibility and traceability is provided.

8. The improved marking system of claim 7, wherein coils are located inside, outside and upside down relative to orthogonal planes bisecting the stent, whereby at least proximal, middle and distal placement is used.

9. A process for using marking coil medical device systems, comprising, in combination:
providing at least a marking coil device affixed to a stenting member capable of being located on different long loops of the stenting member; and
affixation is accomplished via a secondary attachment mechanism or directly to the stenting member;
wherein an at least two marking coils are attached on different or separate long loops.
